(19) United States
(12) Patent Application Publication
(10) Pub. No.: US 2015/0065689 A1
(43) Pub. Date: Mar. 5, 2015

(54) SINGLE STEP FRACTIONATION METHOD

(71) Applicant: DR. REDDY'S LABORATORIES LIMITED, Qutubullapur (IN)

(72) Inventors: Ashish K Patra, Bhubaneswar (IN); Venkata Ramireddy Yeturu, Hyderabad (IN); Jaby Jacob, Kottayam (IN)

(73) Assignee: DR. REDDY'S LABORATORIES LIMITED, Qutubullapur (IN)

(21) Appl. No.: 14/391,116

(22) PCT Filed: Apr. 8, 2013
(86) PCT No.: PCT/IB2013/052785

§ 371 Date: Oct. 7, 2014

(30) Foreign Application Priority Data
Apr. 10, 2012 (IN) 1431/CHE/2012

(51) Int. Cl. C07K 14/505; C07K 1/20

(52) U.S. Cl. CPC C07K 14/505; C07K 1/20
USPC 530/380

(57) ABSTRACT

Single step method of enriching highly sialylated variant/s of a protein by use of a strong ion exchange chromatography support and a pH gradient in absence of any salt gradient.

### RELATED APPLICATION

This application is related to and takes priority from Indian Provisional Application 1431/CHE/2012 filed 10 Apr. 2012 and is herein incorporated in its entirety.

### BACKGROUND

Aspects of the present invention relate to a single step fractionation method of highly sialylated variant/s of a protein wherein the method comprises use of a strong ion exchange chromatography support and a pH gradient for elution of the said variant/s.

Darbepoetin (U.S. Pat. No. 7,217,689) is an erythropoiesis stimulating protein. Production of recombinant darbepoetin typically leads to accumulation of heterogeneously sialylated variants of the molecule. A direct correlation between the extent of sialylation and serum half-life of darbepoetin has been established (Egirie et. al Oncology, Vol. 16, 2002, 13-22). Darbepoetin composition comprising higher sialylated variants exhibit enhanced in vivo half-life and therefore efficacy, as compared to low sialylated variants. Hence, it is important to fractionate and enrich for higher sialylated variants of the protein.

Different chromatography based methods have been described for separation and enrichment of appropriate darbepoetin variants, in particular highly sialylated variants. However, a significant drawback in the methods described in the prior art are the multistep nature of the fractionation methods.

EP1428878 discloses a method of purifying isoforms of erythropoietin by using at least two anion exchange chromatographic steps separated by one or more chromatographic steps distinct from anion exchange mode. It additionally suggests use of at least one acidic wash step in the anion exchange steps to remove low sialylated forms of the protein.

U.S. Pat. No. 7,012,130 & EP1127063 discloses a multi step method of purifying recombinant human erythropoietin from culture supernatant, by hydrophobic interaction chromatography, anionic exchange chromatography, cationic exchange chromatography; and molecular exclusion chromatography.

US20110098452 describes a multi step method for purifying low pI isoforms of darbepoetin utilizing at least one cation exchange chromatography in flow through mode, and additional chromatographic steps, which could be anion exchange or mixed mode chromatography.

Even though, several techniques are described in the art, they generally multi-step fractionation methods that result in low yields, and are generally cumbersome in nature. Therefore, there is a need for development of simpler fractionation methods that require fewer steps for enrichment of highly sialylated variants of a protein.

The primary object of the present invention is to provide a single step fractionating method for separating highly sialylated variants of an erythropoiesis stimulating protein wherein the method comprises, binding the protein preparation to a strong ion exchange support, and enriching the highly sialylated variants of the protein by using pH gradient elution. A further object of the present invention is to provide a single step fractionation method for separating highly sialylated variants of an erythropoiesis stimulating protein on a strong ion exchange support, wherein no salt gradient is used for fractionation. A further object of the present invention is to provide a single step method for enriching highly sialylated variants of a protein without use of any in-process wash step.

### SUMMARY

Aspects of the present disclosure provide a rapid single step method for fractionation of highly sialylated variants of a protein. The method comprises binding the protein preparation to a strong ion exchange support, and enriching said highly sialylated variants of the protein by using pH gradient elution at a constant salt concentration.

### BRIEF DESCRIPTION OF THE DRAWINGS

FIG. 1 Illustration of darbepoetin variant fractionation using a pH gradient at constant salt concentration as described in Example 1.

FIG. 2 Magnified illustration of Region II described in FIG. 1.

FIG. 3 Magnified illustration of Region I described in FIG. 1.

FIG. 4 Isoelectric focusing of fractionated highly sialylated variants as described in Example 1.

FIG. 5 Western Blot of Isoelectric focused gel described in FIG. 4 with polyclonal anti-darbepoetin antibody.

### DETAILED DESCRIPTION

In one embodiment, the invention provides a method for fractionation of highly sialylated variants of an erythropoiesis stimulating protein comprising,

a) loading a clarified cell culture broth comprising a mixture of differentially sialylated erythropoiesis stimulating protein on to a strong ion exchange resin,
and
b) eluting the bound protein using a pH gradient and at a constant salt concentration, wherein the eluate is enriched in highly sialylated variants of the protein.

In a further embodiment the ion exchange chromatography is devoid of any inprocess wash step.

In another embodiment, the invention provides a method of fractionation of highly sialylated variants of an erythropoiesis stimulating protein comprising, loading a clarified cell culture broth comprising a mixture of differentially sialylated erythropoiesis stimulating protein on to a strong ion exchange resin wherein the column is pre equilibrated with a buffer at near neutral pH and a suitable salt concentration

and
b) eluting the bound protein using a pH gradient and at a constant salt concentration, wherein the eluate is enriched in highly sialylated variants of the protein

In a further embodiment the ion exchange chromatography is devoid of any in-process wash step.

In another embodiment of the invention, the erythropoiesis stimulating protein is darbepoetin.

In yet another embodiment of the invention, the strong ion exchange resin is an anion exchange resin.

In a further embodiment of the invention, the ion exchange resin is pre-equilibrated with a buffer comprising 90 mM sodium chloride. In yet another embodiment, the pH of the equilibration buffer is between the isoelectric point of the highly sialylated variant of interest and neutral pH.

In yet another embodiment of the invention the conductivity of the elution buffer is equal to that of the equilibration buffer.

In yet another embodiment of the invention the fractionation is performed using a highly pressure liquid chromatography (HPLC).

In yet another embodiment of the invention, the pH gradient is established by mixing neutral and acidic pH buffer at a predefined rate such that a linear gradient between near neutral (about 7.5) and acidic (about 2.0) is established.

In a further embodiment of the invention, the erythropoiesis stimulating protein is darbepoetin.

The term ‘highly sialylated variant/s’ or ‘highly sialylated protein’ in the context of the present invention refers to a protein, which contains at least about 18 or more sialic acid moieties attached to the protein.

Certain specific aspects and embodiments of the invention are more fully described by reference to the following examples, being provided only for purposes of illustration. These examples should not be construed as limiting the scope of the invention in any manner.

### EXAMPLE 1

### Sample Preparation

Expression of the darbepoetin was accomplished as described in US20110098452, which is incorporated herein as reference. Harvested cell culture was clarified by centrifugation to obtain CCCB (clarified cell culture broth).

### Instrumentation and Blank

Water Alliance HPLC system housing a strong anion exchange support was used. The differentially eluted variants detected using a PDA detector by measuring UV absorbance at 280 nm.

The HPLC profiles for the test samples were analyzed by integrating peaks after subtracting respective buffer blanks.

### Single Step Fractionation by Strong Anion Exchange Chromatography

ProPac® SAX-10 (Dionex) analytical column with a column volume of about 3 ml was pre-equilibrated with 10 mM phosphate, 90 mM NaCl buffer (pH 7.3), followed by loading neat untreated clarified cell culture broth obtained post cell culture on to it. An equilibration salt concentration of 90 mM sodium chloride minimized binding of low sialylated variants of the protein to the column. A linear pH gradient was established by mixing the acidic buffer with the neutral buffer (Table 1) at a rate of about 5% percent per minute (Table 2), and flow rate of about 1 ml/minute was maintained. Salt concentration of 90 mM sodium chloride, equivalent to that of the equilibration buffer was maintained in the acidic and neutral buffer.

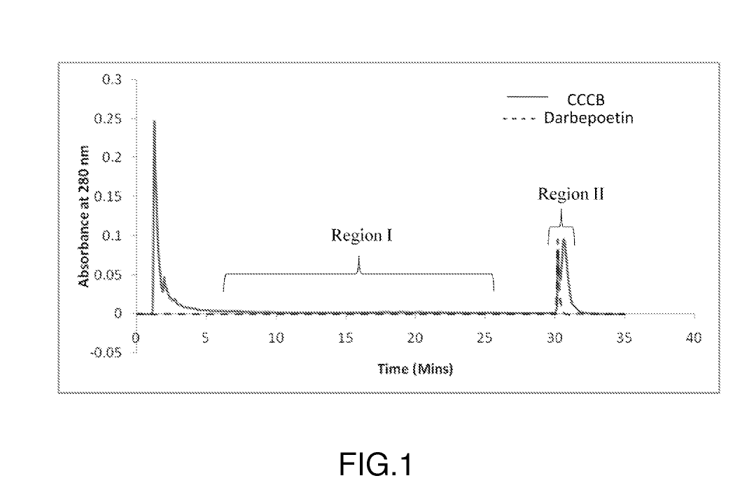

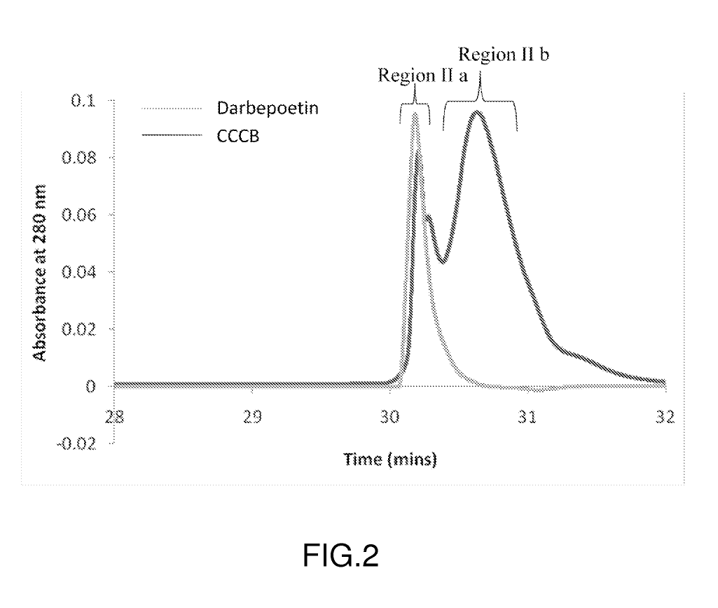

Darbepoetin containing about 18-22 sialylic acid moieties per molecule was used as a control and run under identical conditions. FIG. 1 and FIG. 2 (magnification of total Region II of FIG. 1) illustrates the chromatogram demonstrating concurrent elution of darbepoetin (grey line) with Region IIA corresponding to the highly sialylated variants of darbepoetin from clarified cell culture broth (dark line) at about 30 min under given conditions. The pH of the elution buffer was altered from about 7.3 to about 2.5. Elution of highly sialylated variants was obtained at a pH of around 2.5. Region IIB was determined to be non-proteinaceous.

TABLE 1
Buffers used for fractionation of sialylated protein

| S. No | Buffer | Composition |
|---|---|---|
| 1 | Neutral Buffer | 10 mM phosphate, 90 mM NaCl, pH 7.3 |
| 2 | Acidic Buffer | 5 mM phosphoric acid, 90 mM NaCl, pH |
| | | ~2.5 |

TABLE 2
Rate of mixing of neutral and acidic buffers of Table 1

| | Flow rate | Mobile phase | |
|---|---|---|---|
| Time (min) | (mL/min) | Neutral Buffer % | Acidic Buffer % |
| 0-4 | 1.0 | 100 | 0 |
| 4-24 | 1.0 | 0 | 100 |
| 24-28 | 1.0 | 0 | 100 |
| 28-29 | 1.0 | 100 | 0 |
| 29-35 | 1.0 | 100 | 0 |

A linear pH gradient was attained by mixing at the rate of 5% per minute of the acidic buffer to the neutral buffer. The salt concentration was maintained constant at 90 mM sodium chloride.

A correlation between the rate of gradient formation, flow rate and expected elution time of the desired highly sialylated variant is expected. In other words, a slower gradient rate may demand a slower flow rate and therefore the expected variant may elute at a later time point.

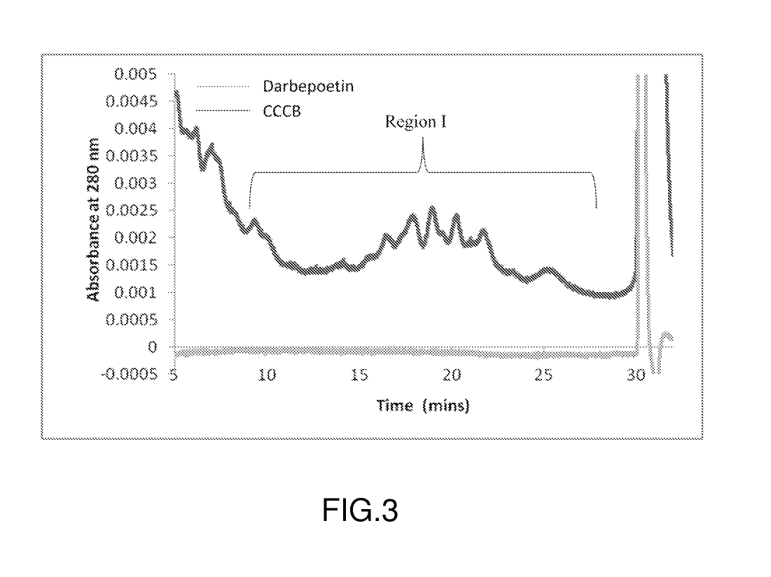

FIG. 3 is magnification of the Region I of FIG. 1, and illustrates the importance of the pH gradient. Use of the gradient enabled fractionation and elution of any low sialylated variant/s of the protein that bound to the column. Whereas, use of a salt concentration of about 90 mM led at the equilibration step lead to diminished binding of low sialylated variants of the protein to the column, and elution with a pH gradient at similar salt concentration led to preemptive elution of low sialylated variants of the protein, thus enriching the highly sialylated variant/s.

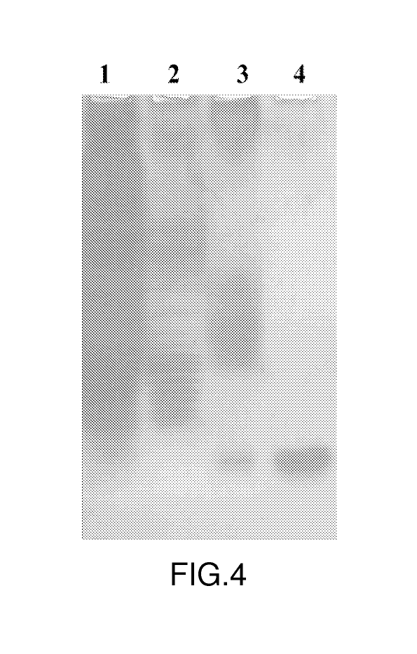

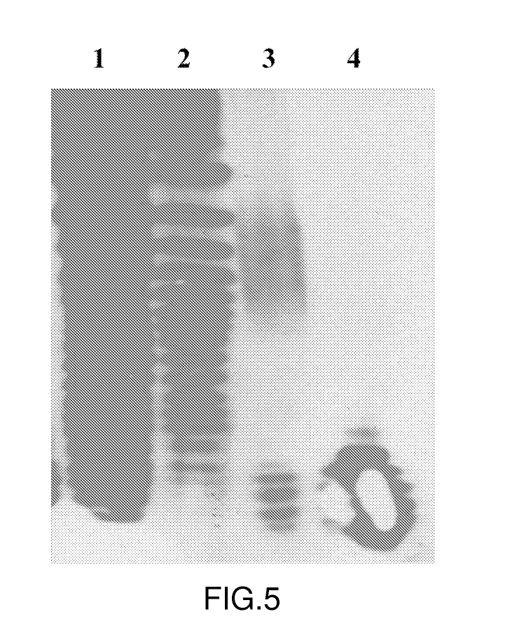

Isoelectric focusing and western blot analysis (FIGS. 4 and 5 respectively) was used to determine enrichment of highly sialylated variant of darbepoetin. Lane 1 and 2 represent the CCCB and flow-through from the column respectively. Lane 3 represents the enriched highly sialylated variant obtained post elution using the pH gradient at constant salt concentration. Lane 4 represents darbepoetin used as control.

We claim:

1. A method for fractionation of highly sialylated variants of an erythropoiesis stimulating protein comprising:
a) loading a clarified cell culture broth comprising a mixture of differentially sialylated erythropoiesis stimulating protein on to an ion exchange resin, and
b) eluting the bound protein using a pH gradient at a constant salt concentration, wherein the eluate is enriched in highly sialylated variants of the protein.

2. A method according to claim 1, wherein the erythropoiesis stimulating protein is darbepoetin.

3. A method according to claim 1, wherein the ion exchange resin is a strong ion exchange resin.

4. A method according to claim 3, wherein the strong ion exchange resin is an anion exchange resin.

5. A method according to claim 4, wherein the strong ion exchange resin is pre-equilibrated with a buffer comprising 90 mM sodium chloride and wherein the pH of the equilibration buffer is between the isoelectric point of the sialylated variant and the neutral pH.

6. A method according to claim 1, wherein the pH gradient is a linear pH gradient established between about 7.5 and about 2.0.

7. A method according to claim 1, wherein the ion exchange chromatography is devoid of any in process wash step.

8. A method according to claim 1, wherein the conductivity of the elution buffer is equal to that of the equilibration buffer.

9. A method according to claim 1, wherein the fractionation is performed using a high pressure liquid chromatography.

10. A method according to claim 1, wherein highly sialylated variants of an erythropoiesis stimulating protein contains at least about 18 or more sialic acid moieties attached to the protein.
